(19) United States
(12) Patent Application Publication
(10) Pub. No.: US 2024/0058232 A1
(43) Pub. Date: Feb. 22, 2024

(54) Composition Comprising Calcium Orthophosphate and a Bioactive Glass Comprising Fluorine

(71) Applicant: Queen Mary University of London, London (GB)

(72) Inventors: Robert Hill, London (GB); Melissa Tiskaya, London (GB); David Geoffrey Gillam, London (GB)

(73) Assignee: Queen Mary University of London, London (GB)

(21) Appl. No.: 18/268,933

(22) PCT Filed: Dec. 22, 2021
(86) PCT No.: PCT/GB2021/053407

§ 371 Date: Jun. 21, 2023

(30) Foreign Application Priority Data
Dec. 22, 2020 (GB) 2020423.6

(51) Int. Cl. A61K 8/21; A61K 8/25; A61K 8/24; A61Q 11/00

(52) U.S. Cl. CPC A61K 8/21; A61K 8/25; A61K 8/24; A61Q 11/00; A61K 2800/412

(57) ABSTRACT

A composition comprising a mixture of a calcium orthophosphate and a bioactive glass comprising fluorine is useful in a non-aqueous toothpaste.

The present invention relates to a composition comprising a mixture of a calcium orthophosphate and a fluorine containing bioactive glass. The composition can be used in a non-aqueous toothpaste.

### BACKGROUND OF THE INVENTION

Bioactive glasses are attractive additives for toothpastes for treating dentine hypersensitivity and for inhibiting tooth decay and promoting re-mineralization they have been used for almost twenty years. The first step in the degradation of a bioactive glass is the ion exchange of Na⁺ and Ca²⁺ in the glass for H⁺ ions in the external solution. This ion exchange increases the pH of the saliva and acts to inhibit the dissolution of the tooth mineral. In addition as the glass dissolves it releases Ca²⁺ and orthophosphate (PO₄³⁻) ions, which supersaturate the external media and result in the formation of a hydroxyapatite like phase. In the mouth bioactive glasses can be used to put back the apatite mineral lost from teeth as a result of acid dissolution either by cariogenic bacteria or as a result of consumption of acidic beverages which is termed “acid erosion”.

Recently fluorine containing bioactive glasses have been developed that release F⁻ ions in addition to Ca²⁺ and PO₄³⁻ ions. These glasses form fluorapatite (Ca₅(PO₄)₃F which is much more acid durable than hydroxyapatite (Ca₅(PO₄)₃OH and dissolves at a pH of 25 about 4.5 rather than 5.5 for hydroxyapatite. These new bioactive glasses with their slow release of fluoride have been developed commercially for toothpastes.

The problem, of fluoride ions reacting with Ca²⁺ and phosphate ions in aqueous toothpastes is a significant problem in formulations containing calcium and phosphate. Fluorapatite like phases can form, whilst in formulations with calcium alone fluorite (CaF₂) can form. For example in aqueous toothpastes based on amorphous calcium phosphates (ACP) the ACP can take up F⁻ ions and form a phase close in structure to fluorapatite, which reduces the concentration of free fluoride ions available [Chen et al. Characterisation of Casein Phosphopeptide Amorphous calcium phosphate based products with and without sodium fluoride. International Journal of Development Research 07 17221−17224, (2017). Reynolds Fluoride Composition and Methods of Dental Mineralisation US 966894562].

It has been found that this type of problem can be overcome by using a fluorine containing bioactive glass, rather than a free source of fluoride ions.

In the mouth the salivary fluoride concentrations from toothpastes containing soluble fluorides such as NaF decay rapidly and exponentially with time [Duckworth et al Fluoride in Saliva and Plaque Following Use of Fluoride-containing Mouthwashes J DENT RES 1987 66: 1730] as a result of salivary flow in the mouth to such an extent that the [F⁻] concentration falls below a therapeutic value typically in under 90 minutes after tooth brushing. It is widely recognized that low concentrations of F⁻ ions in saliva are effective in preventing caries and promoting tooth re-mineralization. Ten Cate [Ten Cate Review on fluoride, with special emphasis on calcium fluoride mechanisms in caries prevention Eur J Oral Sci 1997; 105: 461−465] and others have recognized the advantage of a slow sustained release of fluoride over the time interval between tooth brushing (10−12 hours). Fluorine containing bioactive glasses can achieve this. The small bioactive glass particles adhere to the tooth and dissolve over 10−12 hours releasing F⁻ ions in addition to Ca²⁺ and PO₄³⁻ ions and forming fluorapatite. Whilst it is generally accepted that fluoride concentrations in saliva decay exponentially because of salivary flow in the mouth. It has now been found through recent data that some of the decay of F⁻ observed in salivary fluoride clearance studies may also be due to loss of F⁻ through formation of insoluble CaF₂ (particularly at high [F⁻] and low pH) and by the consumption of F⁻ ions in the formation of Fluorapatite particularly at low [F⁻] less than 45 ppm. In addition at neutral pH there may also be simple ion exchange of the OH⁻ ion of Hydroxyapatite for F⁻ ions in solution.

Fluorine containing bioactive glasses in particulate form (in contrast to soluble fluoride sources such as NaF) attach to the hard and soft tissues in the mouth and release their fluoride slowly as advocated by Ten Cate.

There are however some disadvantages to Fluorine containing Bioactive Glasses. These include:

1) The speed of apatite formation and the amount of apatite that forms increases with the P₂O₅ content of the glass. Ideally we would like a glass that released fluoride plus Ca²⁺ and orthophosphate (PO₄³⁻) in a molar ratio close to that of apatite (Ca₅(PO₄)₃OH i.e. 5:3 or 1.67 but it is difficult to achieve a Ca:P molar ratio of above 3 before the glass starts to crystallize. Better still would be a fluorine containing bioactive glass that released Ca²⁺ and orthophosphate (PO₄³⁻) and fluoride in a molar ratio close or slightly higher than that of fluorapatite (Ca₅(PO₄)₃F i.e. 5:3:1. Crystallization of the glass generally reduces its reactivity, its ability to release ions and to form apatite.

2) Bioactive glasses are relatively expensive components for toothpastes. The cost is acceptable for dentine hypersensitivity toothpastes where consumers will pay a higher price. However, the cost is too high for many consumers and particularly for toothpastes targeted towards anti caries, or re-mineralising toothpastes.

It has now been found that one option to address these problems would be to use a much higher fluorine content glass than is currently used in existing fluorine containing bioactive glass toothpastes and to blend this glass with a source of an inexpensive calcium phosphate. The objective is to obtain most of the orthophosphate (PO₄³⁻) and the calcium from the calcium phosphate salt, rather than from the glass. By using a higher fluorine content glass with a calcium phosphate it may be possible to get the same efficacy but with a smaller amount of expensive glass.

### SUMMARY OF THE INVENTION

In accordance with a first aspect of the present invention there is provided a composition comprising a calcium orthophosphate and a bioactive glass comprising fluorine.

Preferably, the calcium orthophosphate is a hydroxyl deficient calcium orthophosphate.

Preferably, the calcium orthophosphate is a tricalcium phosphate with a calcium to phosphorus molar ratio of 1.25:1 to 1.75:1. More preferably, the calcium orthophosphate is a hydroxyl deficient tricalcium phosphate with a calcium to phosphorus molar ratio of 1.25:1 to 1.75:1.

Preferably, the bioactive glass has a fluoride content expressed as CaF₂ or SrF₂ of about 1 to about 30 mole percent.

Preferably, the bioactive glass has a fluoride content of about 5 to about 25 mole percent. More preferably, the bioactive glass has a fluoride content of about 8 to about 23 mole percent.

Preferably, the composition comprises about 0.1% to about 15% by weight bioactive glass. More preferably, the composition comprises about 0.1% to about 5% by weight bioactive glass. Most preferably, the composition comprises about 1% to about 3% by weight bioactive glass.

Preferably, the composition comprises about 0.1% or more by weight calcium orthophosphate. More preferably, the composition comprises about 0.76% or more by weight calcium orthophosphate. More preferably, the composition comprises about 1% or more by weight calcium orthophosphate.

Preferably, the composition comprises about 16% or less by weight calcium orthophosphate. More preferably, the composition comprises about 6% or less by weight calcium orthophosphate.

Preferably, the composition comprises an amount by weight of calcium orthophosphate, which is between the lower and upper amounts indicated above. In this regard, preferably, the composition comprises about 0.1% or about 0.76% or about 1% to about 16% or 6% by weight calcium orthophosphate.

Preferably, the D50 particle size of the calcium orthophosphate is 12 microns or less.

Preferably, the D50 particle size of the bioactive glass is 12 microns or less.

Preferably, the D90 particle size of the calcium orthophosphate is 60 microns or less.

Preferably, the D90 particle size of the bioactive glass is 60 microns or less.

Preferably, the bioactive glass contains substantially no phosphate. Preferably, the bioactive glass contains substantially no calcium oxide.

Preferably, the calcium orthophosphate is in the form of a calcium deficient apatite that contains substantially no hydroxyl ions.

Preferably, the calcium orthophosphate is in the form of a tricalcium phosphate.

Preferably, the calcium orthophosphate is in the form of an amorphous calcium phosphate.

In a further aspect, the invention provides a composition of the invention for use in the manufacture of a toothpaste. Preferably, the toothpaste is a non-aqueous toothpaste.

In a further aspect, the invention provides a toothpaste comprising a composition of the invention.

In a further aspect, the invention provides a composition or toothpaste for use in the treatment or prevention of dental caries.

In a further aspect, the invention provides a method for making a toothpaste, which comprises the step of mixing a calcium orthophosphate and a bioactive glass comprising fluorine to formulate a composition of the invention.

### BRIEF DESCRIPTION OF THE DRAWINGS

The invention will now be further described with reference to the accompanying drawings in which:

FIG. 1 shows an XRD pattern of tricalcium phosphate. The pattern matches that for crystalline beta Tricalcium Phosphate;

FIG. 2 shows a ³¹P MAS-NMR spectrum for the tricalcium phosphate selected. The spectrum matches that for beta tricalcium phosphate;

FIG. 3 shows the XRD pattern of Table 1 Glass 13 before and after immersion in 0.1 M acetic acid pH 4.5;

FIG. 4 shows the XRD pattern of a 1:4 weight ratio Table 1 Glass 13:TCP mixture after up to 24 hours of immersion in 0.1 M acetic acid pH 4.5;

FIG. 5 shows the XRD pattern of a 2:3 weight ratio Table 1 Glass 13:TCP mixture after 0 and 24 hours of immersion in 0.1 M acetic acid pH 4.5;

FIG. 6 shows the XRD pattern of a 2.5:2.5 weight ratio Table 1 Glass 13:TCP mixture after 0 and 24 hours of immersion in 0.1 M acetic acid pH 4.5;

FIG. 7 shows ³¹P MAS-NMR spectra of a 2:3 weight ratio Table 1 Glass 13:TCP mixture before and after immersion in 0.1 M acetic acid pH 4.5;

FIG. 8 shows ¹⁹F MAS-NMR spectra of a 2:3 weight ratio Table 1 Glass 13:TCP mixture before and after immersion in 0.1 M acetic acid pH 4.5;

FIG. 9 shows the XRD pattern of Table 1 Glass 14 before and after immersion in 0.1 M acetic acid pH 4.5;

FIG. 10 shows the XRD pattern of a 2:3 weight ratio Table 1 Glass 14:TCP mixture before and after immersion in 0.1 M acetic acid pH 4.5;

FIG. 11 shows ³¹P MAS-NMR spectra for a 2:3 weight ratio Table 1 Glass 14:TCP mixture before and after immersion in 0.1 M acetic acid pH 4.5;

FIG. 12 shows the ¹⁹F MAS-NMR spectra of a 2:3 weight ratio Table 1 Glass 14:TCP mixture before and after immersion in 0.1 M acetic acid pH 4.5;

FIG. 13 shows ³¹P MAS-NMR spectra of a 2:3 weight ratio Table 1 Glass 15:TCP mixture before and after immersion in 0.1 M acetic acid pH4.5;

FIG. 14 shows ¹⁹F MAS-NMR spectra of a 2:3 weight ratio Table 1 Glass 15:TCP mixture before and after immersion in 0.1 M acetic acid pH4.5;

FIG. 15 shows XRD patterns of 2:3 weight ratio mixtures of Table 1 Glass 4:TCP at 0 h, 6 h and 24 h;

FIG. 16 shows ³¹P MAS-NMR spectra of a 2:3 weight ratio Table 1 Glass 4:TCP mixture before and after immersion in 0.1 M acetic acid pH 4.5;

FIG. 17 shows ¹⁹F MAS-NMR spectra of a 2:3 weight ratio Table 1 Glass 4:TCP mixture before and after immersion in 0.1 M acetic acid pH 4.5;

FIG. 18 shows the XRD pattern of a 2:3 weight ratio Table 1 Glass 2:TCP mixture before and after immersion in 0.1 M acetic acid pH 4.5;

FIG. 19 shows ³¹P MAS-NMR spectra of a 2:3 weight ratio Table 1 Glass 2:TCP mixture before and after immersion in 0.1 M acetic acid pH 4.5;

FIG. 20 shows ¹⁹F MAS-NMR spectra of a 2:3 weight ratio Table 1 Glass 2:TCP mixture before and after immersion in 0.1 M acetic acid pH 4.5;

FIG. 21 shows the XRD pattern of a 2:3 weight ratio Table 1 Glass 6:TCP mixture before and after immersion in 0.1 M acetic acid pH 4.5;

FIG. 22 shows ³¹P MAS-NMR spectra of a 2:3 weight ratio Table 1 Glass 6:TCP mixture before and after immersion in 0.1 M acetic acid pH 4.5;

FIG. 23 shows ¹⁹F MAS-NMR spectra of a 2:3 weight ratio Table 1 Glass 6:TCP mixture before and after immersion in 0.1 M acetic acid pH 4.5;

FIG. 24 shows the XRD pattern of a 2:3 weight ratio Table 1 Glass 7:TCP mixture before and after immersion in 0.1 M acetic acid pH4.5;

FIG. 25 shows ³¹P MAS-NMR spectra of a 2:3 weight ratio Table 1 Glass 7:TCP mixture before and after immersion in 0.1 M acetic acid pH 4.5;

FIG. 26 shows ¹⁹F MAS-NMR spectra of a 2:3 weight ratio Table 1 Glass 7:TCP mixture before and after immersion in 0.1 M acetic acid pH 4.5;

FIG. 27 shows the XRD pattern of a 2:3 weight ratio Table 1 Glass 5:TCP mixture before and after immersion in 0.1 M acetic acid pH 4.5;

FIG. 28 shows ³¹P MAS-NMR spectra of a 2:3 weight ratio Table 1 Glass 5:TCP mixture before and after immersion in 0.1 M acetic acid pH 4.5;

FIG. 29 shows ¹⁹F MAS-NMR spectra of a 2:3 weight ratio Table 1 Glass 5:TCP mixture before and after immersion in 0.1 M acetic acid pH 4.5;

FIG. 30 shows the pH rises as a result of glass degradation for 2:3 weight ratio mixtures in 0.1 M acetic acid;

FIG. 31 shows the XRD pattern of a 2:3 weight ratio NaF:TCP mixture before and after immersion in 0.1 M acetic acid pH 4.5;

FIG. 32 shows ³¹P MAS-NMR spectra of a 2:3 weight ratio NaF:TCP mixture before and after immersion in 0.1 M acetic acid pH 4.5;

FIG. 33 shows ¹⁹F MAS-NMR spectra of a 2:3 weight ratio NaF:TCP mixture before and after immersion in 0.1 M acetic acid pH 4.5; and

FIG. 34 shows fluoride concentrations in solution after immersion in 0.1 M acetic acid at pHs of 4.0 and 4.5.

### DETAILED DESCRIPTION OF THE INVENTION

It will be appreciated that aspects, embodiments and preferred features of the invention have been described herein in a way that allows the specification to be written in a clear and concise way. However, unless circumstances clearly dictate otherwise, aspects, embodiments and preferred features can be variously combined or separated in accordance with the invention. Thus, preferably, the invention provides a device having features of a combination of two or more, three or more, or four or more of the aspects described herein. In a preferred embodiment, a device in accordance with the invention comprises all aspects of the invention.

Within the context of this specification, the word “about” or “approximate” means preferably plus or minus 20%, more preferably plus or minus 10%, even more preferably plus or minus 5%, most preferably plus or minus 2%.

Within the context of this specification, the word “comprises” means “includes, among other things” and should not be construed to mean “consists of only”.

Within the context of this specification, the word “substantially” means preferably at least 90%, more preferably 95%, even more preferably 98%, most preferably 99%.

Within the context of this specification, the composition of the invention is preferably in the form of a physical mixture. Preferably, the composition of the invention is not in the form of a composition having a single crystal phase.

Within the context of this specification, a bioactive glass is a glass that when immersed in a physiological solution forms an apatite like phase. A biologically active (or bioactive) material is one which, when implanted into living tissue, induces formation of an interfacial bond between the material and the surrounding tissue. Bioactive glasses are a group of surface-reactive glasses, which exhibit bioactivity. The bioactivity of these glasses is the result of complex reactions which take place on the surface of the glass under physiological conditions, and which result in the formation of hydroxycarbonated apatite (HCA) on the surface of the glass. The term “bioactive glass” as used herein is intended to encompass bioactive glass-ceramics as well as bioactive glasses. Bioactive glass-ceramics are similar to bioactive glasses but contain a crystalline phase in addition to the glass phase.

Within the context of this specification, network connectivity is the average number of bridging oxygens per silicon in a glass structure. It may be calculated according to Hill and Brauer “Predicting the bioactivity of glasses using the network connectivity or split network models” Journal of Non-Crystalline Solids 357 (2011) 3884−3887.

Within the context of this specification, tricalcium phosphate (TCP) is a calcium phosphate with a molar ratio of Ca to P of 1.5 that has an x-ray diffraction pattern that matches ether JCPDS PDF no, 09469) for beta TCP or JCPDS 29−359) for alpha TCP.

Within the context of this specification, hydroxyapatite (Hap) means a crystalline calcium phosphate with the approximate formula Ca₁₀(PO₄)₆(OH)₂.

Within the context of this specification, fluorapatite means a crystalline calcium phosphate with the approximate formula Ca₁₀(PO₄)₆(F)₂.

Within the context of this specification, an anhydrous or non-aqueous toothpaste means a toothpaste containing less than 2% of water by weight.

Within the context of this specification, D50 refers to a particle size distribution D50, which is also known as median diameter or medium value of particle size distribution, it is the value of the particle diameter at 50% in the cumulative distribution. For example, if D50=12 μm, then 50% of the particles in the sample are larger than 12 μm, and 50% smaller than 12 μm.

Within the context of this specification, D90 refers to a particle size distribution D90. It represents the particle diameter corresponding to 90% cumulative (from 0 to 100%) undersize particle size distribution. In other words, if particle size D90 is 60 μm, 90% of the particles in the tested sample are smaller than 60 μm, or the percentage of particles smaller than 60 μm is 90%.

Mixtures of a bioactive glass having a high fluoride content with nano hydroxyapatite were investigated. The bioactive glass is about £65/kg whilst the nano hydroxyapatite is <£6/Kg. The investigation was carried out to determine whether the bioactive glass could provide the bulk of the fluoride and the slow sustained delivery of fluoride, whilst the hydroxyapatite could provide the bulk of the calcium and phosphate.

Studies with these mixtures surprisingly showed the OH⁻ ion of hydroxyapatite to be rapidly ion exchanged for the F⁻ ions released by the glass at pH7 and converted to fluorapatite, which is more stable than the hydroxyapatite of enamel. This strategy is therefore at least partially flawed as upon an acid challenge it would result in the more soluble hydroxyapatite of the enamel dissolving at the expense of the fluorapatite formed from the hydroxyapatite. This discovery/finding then led to the consideration of other calcium phosphates. Remarkably, only one of these, tricalcium phosphate was found experimentally to be suitable and not to form fluorapatite at pH 7 when mixed with a fluorine containing bioactive glass.

Tricalcium phosphate (Ca₃(PO₄)₂(TCP) does not have a OH⁻ ion within its structure it cannot therefore convert to fluorapatite by an ion exchange mechanism. Note that the ion exchange mechanism is not widely accepted and fluorapatite is thought to form by a dissolution reprecipitation mechanism of hydroxyapatite. Tricalcium phosphate can only form fluorapatite by a dissolution reprecipitation mechanism, which will occur at lower pHs corresponding to caries challenge conditions.

There are many commercially available tricalcium phosphates, but a detailed analysis of them indicates that most of them are actually calcium deficient apatites with a Ca:P molar ratio close to 1.5 and are not crystalline stoichiometric tricalcium phosphates. A suitable commercial tricalcium phosphate that meets the design specification and i) is approved for cosmetics use; ii) has an INCI code; and iii) has an appropriate particle size D50 of approximately 4 microns (comparable with the larger dentinal tubules sizes and therefore also suitable for treating dentine hypersensitivity) and is relatively inexpensive 6−8 euros/kg has been found.

It is desirable to include calcium and phosphate in a toothpaste and in the past this has been achieved by including in the toothpaste a bioactive glass comprising calcium and phosphate. Advantageously, since the calcium and phosphate can be provided largely from the TCP, one advantage provided by the invention is that it is no longer necessary to include a bioactive glass that contains calcium and phosphate, the only requirement being that the glass should release F⁻ ions. This enables a wider possible range of bioactive glass compositions to be used in a toothpaste formulation. These include:
i) Simple SiO₂—Na₂O—NaF glasses
ii) Simple SiO₂—CaO—CaF₂ glasses
iii) SiO₂—CaO—Na₂O—CaF₂ glasses
iv) SiO₂—P₂O₅—CaO—Na₂O—CaF₂ glasses with P₂O₅ contents below those specified in the existing patents.

Because of the slightly larger size of the Sr²⁺ cation relative to the Ca²⁺ cation strontium can replace calcium both in bioactive glasses [WO2007/144662] and in the apatite crystal lattice [O'Donnell et al. “Structural analysis of a series of strontium-substituted apatites” Acta Biomaterialia 4 (2008) 1455−1464].

TCP is only very slight soluble at pH 7 but its solubility increases with reducing pH. Bioactive glasses as they dissolve raise the pH since the first step in the degradation process is the ion exchange of Na⁺ and Ca²⁺ cations in the glass for H⁺ ions in solution. If the glass dissolves too rapidly at low pH it can raise the pH of the acidic solution and this will inhibit the TCP from dissolving. In this situation the TCP will not provide sufficient Ca²⁺ and PO₄³⁻ ions to form much apatite. Thus, without wishing to be bound by theory, it is important that the dissolution rate of the glass and the TCP are approximately matched and the pH increase caused by degradation of bioactive glass is not too rapid. The dissolution rate of the glass depends on factors including the particle size, the network connectivity (NC) and the relative proportion of Na:Ca. Glasses with a higher Na content generally dissolve more quickly than a lower sodium content glass of identical NC.

### EXAMPLES

The following examples of glass compositions shown in table 1 have been investigated:

TABLE 1
Glass Compositions in Mole Percent

| Glass | SiO₂ | P₂O₅ | CaO | SrO | Na₂O | K₂O | CaF₂ | NaF | NC⁺ |
|---|---|---|---|---|---|---|---|---|---|
| 1 | 44.88 | 0.97 | 20.94 | 0 | 23.93 | 0 | 9.28 | 0 | 2.13 |
| 2 * | 33.0 | 6.0 | 34.0 | 0 | 13.0 | 0 | 10.0 | 0 | 2.00 |
| 3 | 34.6 | 5.74 | 23.51 | 0 | 0 | 26.87 | 9.28 | | 2.08 |
| 4 | 40.5 | 5.25 | 29 | | 23.45 | | 1.8 | | 2.29 |
| 5 ^{$} | 50.0 | 6.00 | 17.0 | | 12 | 17 | | | 2.00 |
| 6 ^{$} | 44.0 | | | | 17.0 | 17.0 | | 18.0 | 2.27 |
| 7 ^{$} | 60 | 0 | 7.0 | | 12.0 | 7.0 | 10.0 | | 3.00 |
| 7 | 40 | 0 | 20 | | 25 | | 15 | | 1.75 |
| 9 | 44 | 0 | 20 | | 12 | 12 | 12 | | 2.00 |
| 10 | 34 | 6 | 20 | 2 | 12 | 12 | 14 | | 2.35 |
| 11 | 33 | 6 | 20 | 2 | 12 | 12 | 15 | | 2.30 |
| 12 | 34.6 | 5.74 | 0 | 23.51 | 26.87 | 0 | 9.28 | 0 | 2.08 |
| 13 | 49.48 | 0 | 31.96 | | 8.0 | | 10.31 | | 2.38 |
| 14 | 49.48 | 0 | | 31.96 | 8.0 | | 10.31 | | 2.38 |
| 15 | 49.48 | 0 | 15.98 | 15.98 | 8.0 | 5.15 | 5.15 | | 2.38 |

* Also contains 4.0 Mole % MgO
^{$} Also contains 2 Mole % MgO and ZnO
⁺Calculated according to Hill and Brauer.

All the glasses were produced by a conventional melt quench route by melting the respective oxides carbonates and fluorides in Platinum/Rhodium crucible at temperatures between 1400 and 1550° C. The resulting glass melt was quenched to room temperature by pouring directly into water to produce a granular glass frit which was dried ground and sieved to a <38 micron powder. This powder was then used to generate the glass examples in Table 1.

### Example 1

A mixture of TCP (ex Buddenheim C 73−13) and Glass 13 from Table 1 was prepared by mixing 4 g of the TCP with 1 g of Glass 13. A 150 mg aliquot of this glass was placed in a 150 ml sealed bottle with 100 ml of 0.1 M Acetic acid with the pH adjusted to 4.5 (to mimic caries like conditions). Six such samples were prepared. Each bottle was placed in a shaking incubator at 37° C. The Samples were removed from the incubator after 0.5, 1.0, 2, 4, 6 and 24 hours and filtered off. The resulting powder was dried and analysed by FTIR, XRD and ¹⁹F and ³¹P solid state NMR. The pH of the filtrate was measured and the free fluoride concentration was measured with an ion selective electrode. The solution was analysed by ICP-OES for Si, Ca and P.

For comparison the same experiment was conducted with the TCP alone and Glass 13 alone.

Subsequently the experiment was repeated with different weight ratios of Glass to TCP.

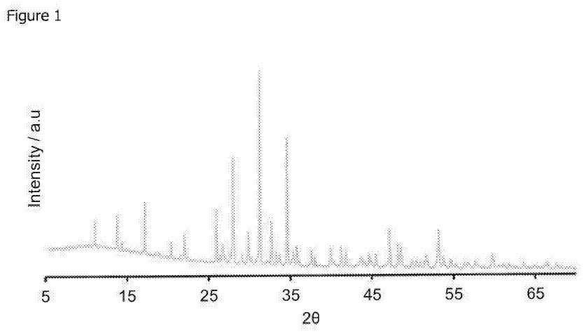

FIG. 1 shows the x-ray diffraction (XRD) pattern of the glass and after immersion in 0.1 M acetic acid pH 4.5. The glass dissolves and CaF₂ precipitates.

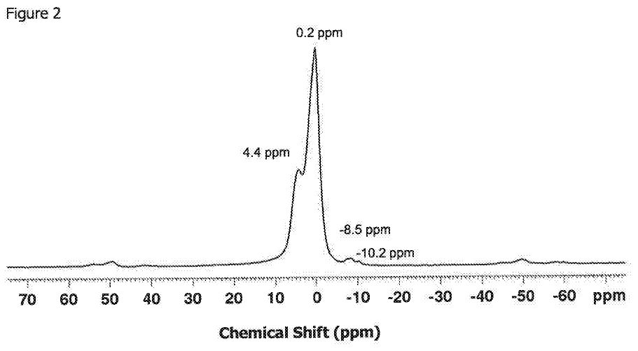

FIG. 2 shows the XRD pattern of the 1:4 and 2.5:2.5 Glass 13:TCP mixtures after 24 hours immersion in 0.1 M Acetic Acid pH 4.5.

Glass 13 dissolves and precipitates CaF₂. It cannot form Fluorapatite in the acetic acid since there is no source of phosphate. When TCP is immersed in 0.1 M Acetic Acid only TCP is detected. When TCP is immersed with Glass 13 it can form fluorapatite and this is seen in FIG. 2. The proportion of Fluorapatite formed increases up 2.5:2.5 and very little TCP is left in the 2:3 mixture.

Table 2 summarises a wide range of Examples if mixtures of bioactive glasses and calcium phosphates that have been investigated together with comments on the phase present and the results achieved with these mixtures.

TABLE 2

| | Glass | TCP | | |
|---|---|---|---|---|
| Glass | Weight | Weight | Phase | |
| Example | (g) | (g) | Present | Comment/Interpretation |
| 13 | 5 | 0 | CaF₂ | No Phosphate Available therefore |
| | | | | forms CaF₂ |
| 13 | 1 | 4 | Small | Phosphate comes from TCP |
| | | | Amount | Dissolution and F from glass |
| | | | FAp | |
| | | | Plus TCP | |
| 13 | 1.5 | 3.5 | Small | Not enough glass in terms of F and |
| | | | Amount | pH rise to form a lot of FAp. |
| | | | FAP | |
| | | | Plus TCP | |
| 13 | 2 | 3 | FAp plus | FAp formed from release of Ca and |
| | | | Trace of | P from TCP and F from glass |
| | | | TCP | |
| 13 | 2.5 | 2.5 | FAp No | TCP totally consumed |
| | | | TCP | |
| 14 | 5 | 0 | SrF₂ | No Phosphate Present and no |
| | | | | apatite formed. |
| 14 | 2 | 3 | Trace of | Fluorapatite mixed Ca/Sr |
| | | | FAp | Strontium stays in solution |
| | | | Lot of TCP | |
| 15 | 5 | 0 | Mixed | No Phosphate to form Apatite |
| | | | Ca/SrF₂ | |
| 15 | 2 | 3 | FAp CaF₂ | Very little mixed Sr Phases Present |
| | | | and TCP | |
| 4 | 2 | 3 | FAP + Lot | Not Enough Fluoride to convert all of |
| | | | of TCP | the TCP to FAp |
| 2 | 2 | 3 | Only FAp | A Perfect combination! |
| | | | No TCP | |
| 6 | 2 | 3 | FApTrace + | Glass dissolves very quickly with its |
| | | | TCP | high sodium content raises pH and |
| | | | | suppresses TCP dissolution? Limits |
| | | | | Phosphate and Calcium release from |
| | | | | TCP. Hence little Apatite formed |
| 5 | 2 | 3 | No Apatite | Some TCP Dissolution No Apatite |
| | | | only TCP | formation with F free glass. Fluoride |
| | | | | more important than just raising the |
| | | | | pH. |
| 7 | 2 | 3 | Small | Large amount of F still in glass |
| | | | amount | phase. Glass not dissolved very |
| | | | CaF₂ plus | much due to high NC of 3.0. |
| | | | TCP and | |
| | | | Glass | |
| NaF | 2 | 3 | Small | High free Fluoride content probably |
| (Not | | | Amount | favours CaF₂ formation at expense |
| Glass) | | | FAp | of FAp plus no glass does not raise |
| | | | Largely | the pH above 4.5 which favours |
| | | | CaF2 some | CaF2 at the expense of fluorapatite. |
| | | | TCP | |

Particle Size

The influence of particle size of the TCP was investigated. Table 3 summarises the particle size data. Instead of using a TCP having a D50 of 4.14 microns an alternative TCP having a D50 of 11.8 microns was mixed with Glass 13 in a ratio of 2:3 by weight. The results obtained were almost identical to those obtained using the TCP having a D50 of 4.14 microns. It was considered that increasing the particle size would decrease the solubility of the TCP but it was surprisingly found that it had little or no influence over this range of particle size.

TABLE 3
Particle size Details for the Tricalcium
Phosphates and a typical glass

| Example | | | | |
|---|---|---|---|---|
| Calcium | | D10 | D50 | D90 |
| Phosphate | Description | (Microns) | (Microns) | (Microns) |
| 1 | βTCP | 0.61 | 4.14 | 15.3 |
| 2 | βTCP | 3.66 | 11.8 | 246 |
| 3 | βTCP | 3.21 | 7.10 | 19.8 |
| | Sintered | | | |
| 4 | Bioactive Glass | 3.77 | 15.0 | 36.7 |

### Example 1: A Phosphate Free Degradable Glass that Forms Fluorapatite with TCP

A mixture of TCP (TCP Example 1 in Table 3) and Glass 13 from Table 1 was prepared by mixing 4 g of the TCP with 1 g of Glass 13. A 150 mg aliquot of this mixture was placed in a 150 ml sealed bottle with 100 ml of 0.1 M Acetic acid with the pH adjusted to 4.5 (to mimic caries like conditions) Six such samples were prepared. Each bottle was placed in a shaking incubator at 37° C. The Samples were removed from the incubator after 0.5, 1.0, 2, 4, 6 and 24 hours and filtered off. The resulting powder was dried and analysed by FTIR, XRD and ¹⁹F and ³¹P solid state NMR. The pH of the filtrate was measured and the free fluoride concentration was measured with an ion selective electrode. For comparison the same experiment was conducted with the TCP alone and Glass 13 alone.

Subsequently the experiment was repeated with different weight ratios of glass to TCP.

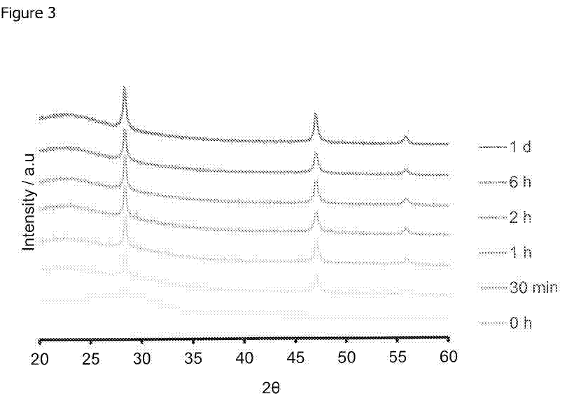

FIG. 3 shows the XRD pattern of Glass 13 and after immersion in 0.1 M Acetic Acid pH 4.5.

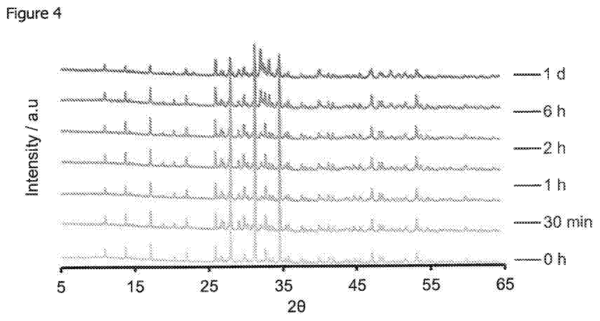

FIG. 4 shows the XRD pattern of the 1:4 weight ratio glass 13:TCP mixtures up to 24 hours of immersion in 0.1 M acetic acid pH 4.5.

The XRD results for the immersed Glass 13 show it to dissolve and to precipitate calcium fluoride CaF₂, evidenced by the diffraction lines at approximately 28 and 47° two theta. Note that this glass cannot form Fluorapatite (Ca₁₀(PO₄)₆F₂) without a source of orthophosphate, since the glass contains no phosphate.

Immersing Tricalcium Phosphate, TCP in 0.1 M Acetic Acid pH4.5 results in dissolution of some of the TCP only and no significant precipitation of new phases. Immersing the 1:4 mixture of glass and TCP results in the formation Fluorapatite, with new diffraction lines for apatite appearing in the diffraction pattern in the range 30−34° two theta at 6 and 24 hours. However the diffraction pattern is still dominated by the diffraction lines of un-dissolved TCP indicating that there is relatively little conversion of the TCP to apatite.

TABLE 4
Measured Concentrations of fluoride in ppm

| | | | | CN-Ca + | CN-Sr + | CN-Ca + |
|---|---|---|---|---|---|---|
| | | CN-Ca | CN-Sr | TCP | TCP | TCP |
| Time | | Glass | Glass | (1:4) | (1:4) | (1.5:3.5) |
| 0 | h | 0.00 | 0.00 | 0.00 | 0.00 | 0.00 |
| 0.5 | h | 15.14 | 38.66 | 9.29 | 8.85 | 9.83 |
| 1 | h | 13.59 | 37.89 | 9.67 | 8.96 | 10.04 |
| 2 | h | 14.26 | 38.66 | 9.25 | 8.92 | 9.39 |
| 4 | h | 12.30 | 39.12 | 9.79 | 8.40 | 9.79 |
| 6 | h | 11.35 | 40.40 | 8.71 | 7.05 | 10.17 |
| 1 | d | 9.07 | 37.59 | 6.45 | 7.45 | 10.48 |

| | | | | | |
|---|---|---|---|---|---|
| Max* | 113 | 85 | 23 | 17 | 34 |

*Maximum possible [F] assuming complete dissolution of the glass.

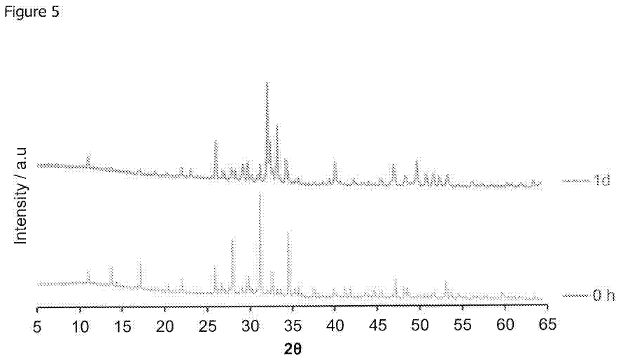

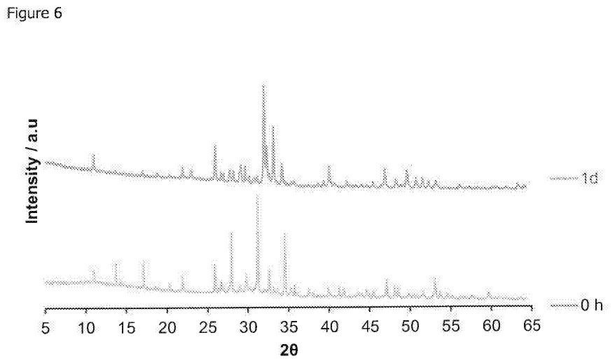

The experiment was repeated with a ratio by weight of Glass 13:TCP of 1.5:3.5, 2:3 and 2.5:2.5. The XRD patterns are shown in FIGS. 5 and 6 for the 2:3 and 2.5:2.5 weight ratios.

In FIGS. 5 and 6 the TCP has dissolved and only fluorapatite is present after 24 hours of immersion. FTIR spectra of samples 3 and 4 showed the characteristic P—O— bands for apatite at 560 and 600 cm⁻¹ wavenumbers, whilst the original TCP showed bands at 540 and 600 cm⁻¹.

It is thought that there was insufficient fluoride released from Glass 13 to convert all the TCP to fluorapatite with the 1:4 weight ratio. As the weight ratio of glass to TCP is increased there will be more fluoride released from the glass plus the pH increase as a result of glass degradation would be greater favouring apatite formation.

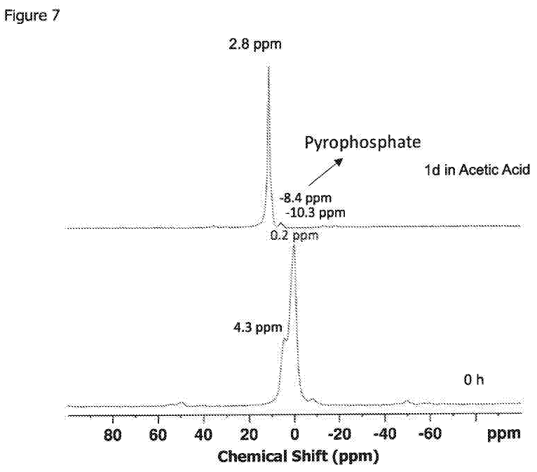

FIG. 7 shows that only apatite is present and all the TCP has been consumed. Apatite has a characteristic sharp peak in its ³¹P MAS-NMR spectra at 2.8 ppm, whilst TCP has two distinct resonances at 4.3 and 0.2 ppm which are present at t=0, but not present at 24 h. There are two small impurity peaks from the TCP present in the original TCP at −8.4 and 10.3 ppm and after immersion which are thought to be due to insoluble pyrophosphates.

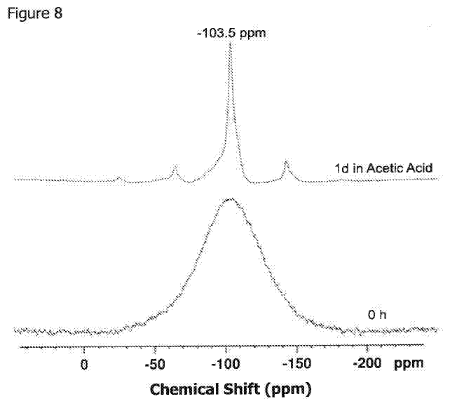

FIG. 8 shows the ¹⁹F MAS-NMR spectra of the 2:3 weight ratio Glass 13 to TCP mixture before and after immersion. The ¹⁹F spectra at 0 h corresponds to the F in the original glass, which is present as mixed F—Ca/Na(n) species in the disordered environment of the glass. Hence the broad peak present. Upon immersion the fluorine is lost from the glass and re-precipitates as Fluorapatite that gives a characteristic resonance at −103.5 ppm corresponding to the F—Ca(3) site in fluorapatite.

TABLE 5
Measured Fluoride concentrations
for 2:3 Mixtures of Glass and TCP

| Glass No | 13 | 14 | 15 | 6 | 2 | 4 | 5 | 7 |
|---|---|---|---|---|---|---|---|---|
| 0 | 0.00 | 0.00 | 0.00 | 0.00 | 0.00 | 0.00 | 0.00 | 0.00 |
| 6 h | | | 16.87 | 10.69 | 13.72 | 3.94 | 0.07 | 7.85 |
| 24 h | 12.06 | 16.98 | 15.44 | 9.95 | 11.34 | 3.19 | 0.05 | 6.47 |

### Example 2: A Strontium Containing Phosphate Free Glass that does not Form Significant Fluorapatite with TCP

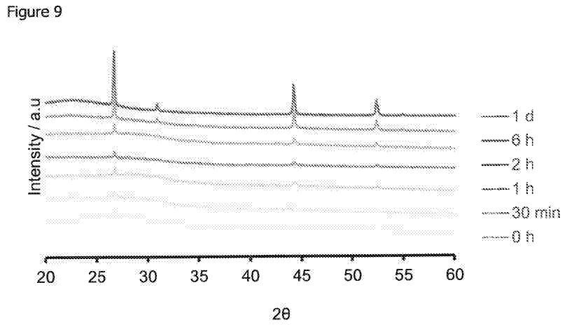

FIG. 9 shows the XRD patterns of Glass 14 after immersion in 0.1 M Acetic acid at pH4.5 from 0 to 24 h. The glass dissolves and forms strontium fluoride SrF₂. The diffraction peaks are shifted to lower two theta values due to the slightly larger size of Sr²⁺ relative to Ca²⁺ (26.7° cf 28.2°).

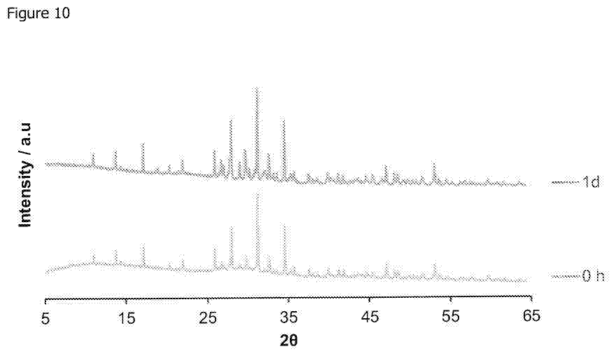

FIG. 10 shows that the TCP remains and that no apatite is formed. This contrast with the equivalent data for the all Ca glass (FIG. 5) where no TCP was detected after immersion and only fluorapatite was present.

This result was unexpected since Sr²⁺ exhibits complete solid solution formation in fluorapatites with Sr²⁺ readily replacing Ca²⁺ ions in the fluorapatite lattice. It was expected that a mixed calcium strontium fluorapatite would form (Ca(₁₀₋ₓ))XSr(PO₄)₆F₂ but this was not observed. The ³¹P MAS-NMR spectra were run before and after immersion in 0.1M Acetic acid pH4.5.

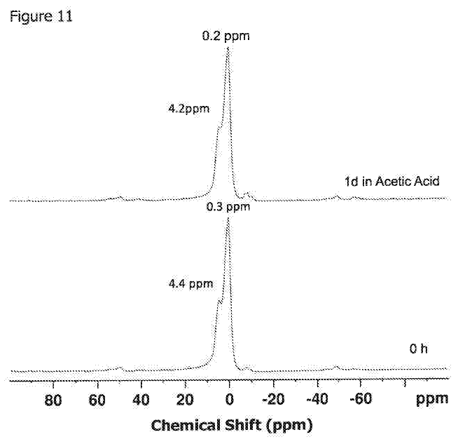

FIG. 11 shows the ³¹P MAS-NMR spectra. The spectrum after immersion is almost identical to that before immersion except the insoluble impurity species thought to be pyrophosphates at −8.4 and 10.3 ppm are increased slightly as a result of some dissolution of TCP.

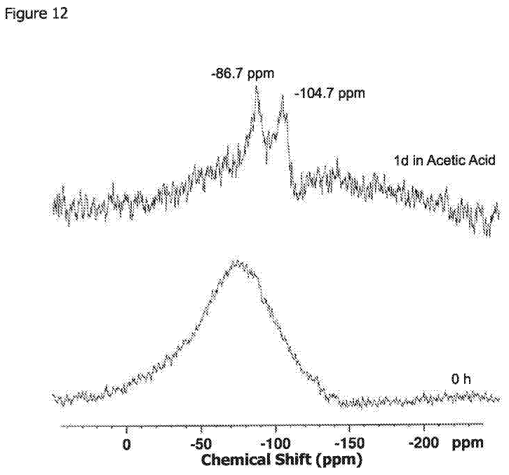

FIG. 12 shows the ¹⁹F spectra of the 2:3 weight ratio mixture of glass 14:TCP before and after immersion in 0.1 M acetic acid pH 4.5.

The ¹⁹F spectra of the initial mixture shows a broad signal shifted slightly to higher chemical shift by comparison with the 2:3 mixture with the all Ca glass corresponding to F—Sr/Na(n) sites in the glass. Upon immersion the F signal is much weaker as evidenced by the much greater signal to noise of the spectrum. A very weak peak at −104.7 ppm corresponding to a F—Ca(n) site in apatite is observed plus a peak at −86.7 ppm, which might be a mixed F—Ca(2)Sr site in apatite. It is important to note that whilst evidence for apatite was detected the amounts detected are exceedingly small. The fluoride concentrations in solution were much higher with this glass than with the equivalent Ca. The pH rise observed however was similar to the Ca equivalent glass. The chemical analysis of the solution indicated that the Sr from the glass had dissolved completely and had not precipitated which is in agreement with the absence of strontium containing crystal phases. The [F⁻] concentration in solution was also much higher than with the Ca equivalent glass and this may in part reflect the higher solubility and solubility products of mixed Ca/Sr fluorapatite and fluorides.

### Example 3: A Mixed Ca/Sr Phosphate Free Glass that Forms Mixed Phases

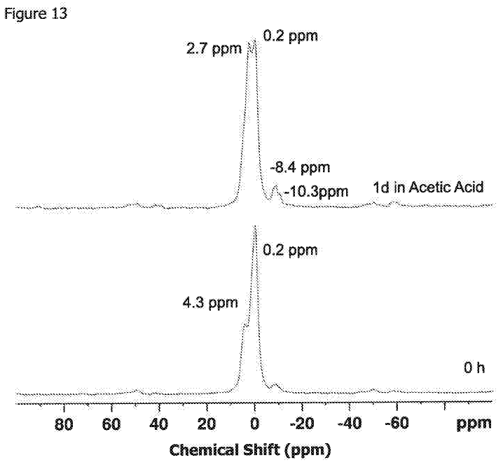

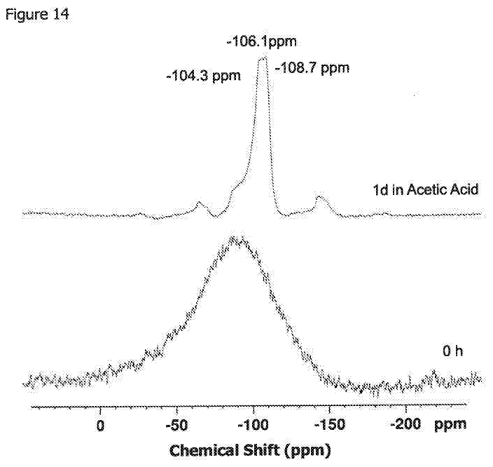

The same experiment was carried out for Glass 15 from Table 1, which is a glass containing equal amounts of Calcium and Strontium in between that of Glass 13 and 14. Only the ³¹P and ¹⁹F spectra are shown for the 2:3 Mixture of Glass 15 to TCP are shown in FIGS. 13 and 14.

FIG. 13 shows a new peak at 2.7 ppm that has formed after 24 h immersion that corresponds with apatite formation. However the principle resonance of TCP is still present at 0.2 ppm indicating that not all the TCP has dissolved and that TCP is still present. The 4.6 ppm resonance of TCP is masked by the presence of apatite.

In FIG. 14, the ¹⁹F MAS-NMR spectra of the mixture at t=0 arises from F in the glass from mixed F—Ca/Sr/Na sites and is broad because of the disordered environments present in the glass. The spectrum after immersion is complex and contains multiple contributions including a −104.3 ppm resonance corresponding to F—Ca(3) sites in Fluorapatite and a −108.7 ppm resonance corresponding to F—Ca(4) sites in CaF₂, plus mixed F—Ca/Sr sites in the range −80−90 ppm. This glass appears to be precipitating largely Calcium fluorapatite and Calcium Fluoride species with smaller amounts of strontium substituted species.

### Example 4: A Low Fluorine Phosphate Containing Glass that Forms Only a Small Amount of Fluorapatite Because of the Low Fluoride Content

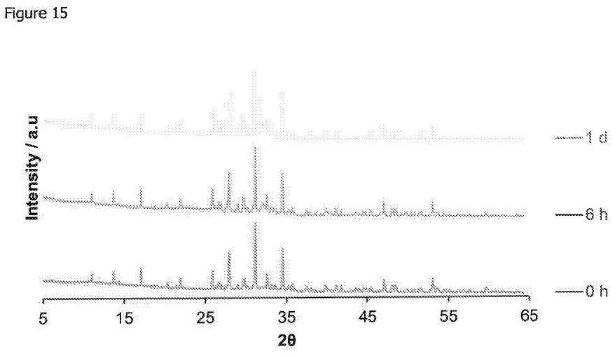

FIG. 15 shows the XRD data for Glass 4 from Table 1 mixed 2:3 with TCP and immersed in 0.1 M Acetic Acid.

There appears to be little change in the diffraction patterns before and after immersion, but closer examination of the two theta range between 31 and 34° two theta indicates a small amount of apatite to have formed.

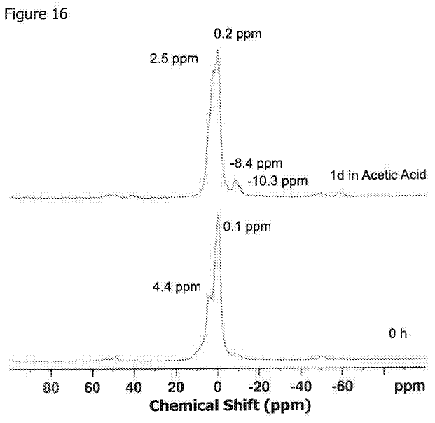

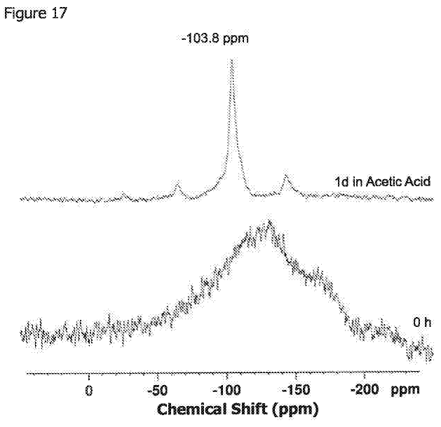

This is confirmed by the ³¹P and ¹⁹F spectra. The ³¹P spectra are shown in FIG. 16. After immersion there is a shoulder at 2.5 ppm in addition to the 0.2 ppm and 4.6 ppm resonance of TCP. The 2.5 pppm resonance is close to that for apatite at 2.7 ppm. The presence of fluorapatite is further confirmed by the ¹⁹F spectra of FIG. 17. Here the glass has a broad spectrum consisting of overlapping signals from F—Ca(n) and F—Can)Na whilst after immersion for 24 h the broad signal from the glass is lost as it dissolves and a new sharp signal appears at −104.8 ppm corresponding to Fluorapatite.

The large amount of TCP left with this glass and the relatively small amount of Fluorapatite formed is probably the result of the low fluorine content (1.8 Mole % CaF₂) of this glass relative to some of the other glass compositions.

### Example 5: A High Fluoride Phosphate Containing Glass that Forms Only Fluorapatite

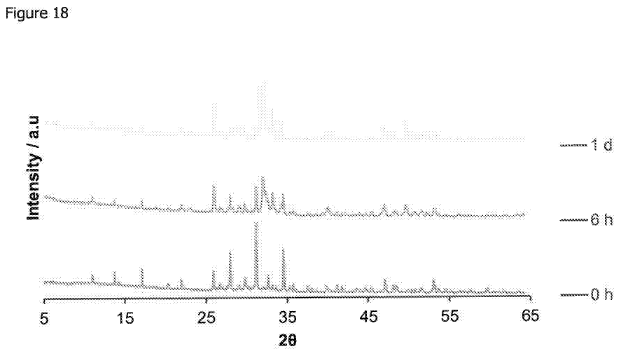

Glass 2 from Table 1 was mixed in a 2:3 ratio with TCP and immersed in 0.1 M Acetic Acid. FIG. 18 shows the XRD patterns. After 6 h there is TCP and apatite present. By 24 h only apatite is present.

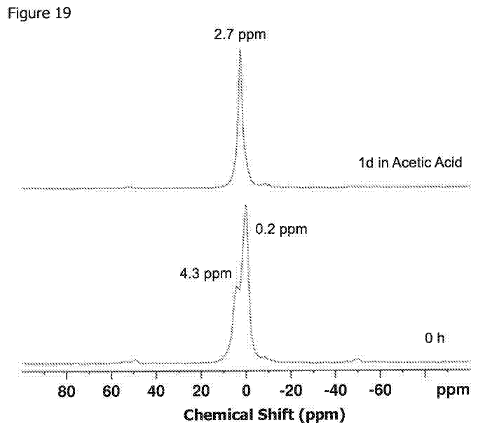

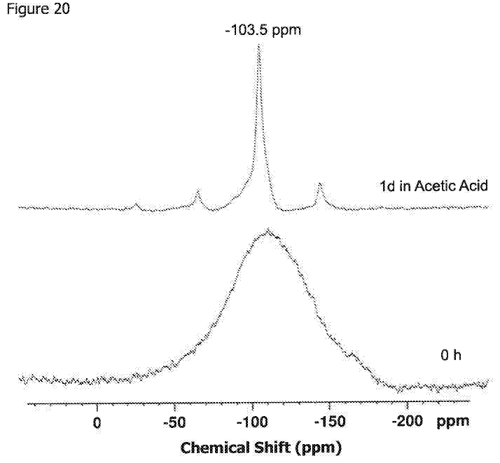

The presence apatite is confirmed by the ³¹P MAS-NMR spectrum (FIG. 19) that shows a single sharp resonance at 2.7 ppm corresponding to fluorapatite and no TCP present. FIG. 20 shows the ¹⁹F MAS-NMR spectra before and after immersion. The ¹⁹F spectra of the un-immersed mixture arises from the glass and again corresponds to mixed F—Ca/Na environments in the glass. This signal is lost by 24 h and a new signal is present at −103.5 ppm corresponding to Fluorapatite formation.

### Example 6: An Alkaline Earth Glass Containing Alkali Metals and Fluoride that Surprisingly Forms Very Little Fluorapatite

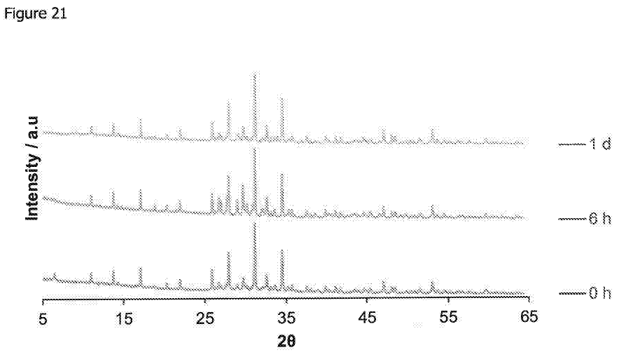

Glass 6 from Table 1 is a calcium free and phosphate free glass containing fluorine. FIG. 21 shows the diffraction pattern of a 2:3 mixture with TCP before and after immersion for 6 h and 24 h. The initial mixture shows the diffraction lines from TCP. No new diffraction lines for apatite are observed after immersion. It was expected that this glass would form fluorapatite upon immersion with TCP with Calcium and Phosphate dissolved from the TCP forming Fluorapatite with the fluoride ions released from the glass.

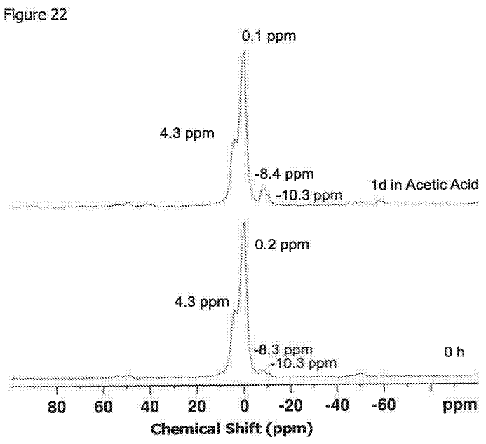

The ³¹P MAS-NMR spectra before and after immersion are shown in FIG. 22 and both before and after immersion only the resonances for TCP are observed at 0.1 and 4.6 ppm with Impurity the peaks in the TCP at 8.6 and 10.4 ppm, which are enhanced in the immersed sample.

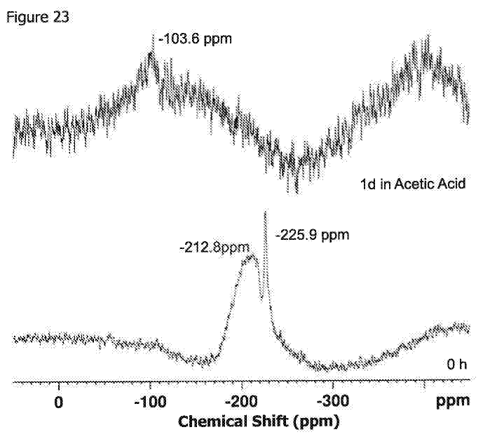

FIG. 23 shows the ¹⁹F Spectra before and after immersion. The initial glass has a broad resonance centred at −212.8 ppm corresponding to disordered F—Na(n) species in the glass. In addition there is a sharp peak at −225.9 ppm corresponding to crystalline NaF, which indicates the glass had crystallised slightly during quenching from the melt during synthesis. This was despite the glass being optically transparent. Upon immersion the broad resonance disappears, as well as the sharp signal for crystalline NaF as these species dissolve. A new peak at −103.6 ppm is formed that corresponds to fluorapatite formation and this is superimposed on a broad background. The Signal to noise (S/N) of this spectrum is poor which would indicate a low fluorine content in the remaining solid. However the measured fluoride concentration in the acetic acid at 6 h and 24 h was 10.69 and 9.95 ppm, which is surprisingly low.

### Example 7: A High Network Connectivity Glass with Fluoride that does not Form Fluorapatite

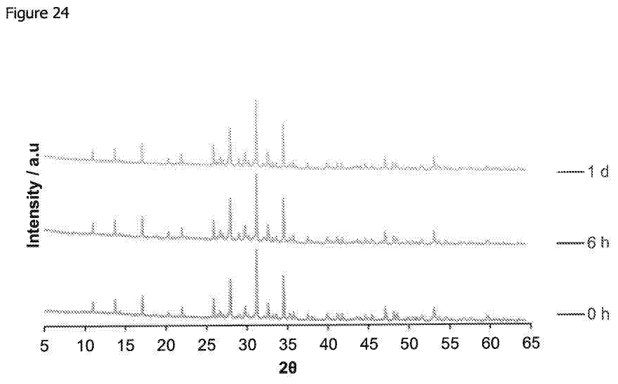

Glass 7 of Table 1 is a high network connectivity glass with a calculated network connectivity (NC) of 3.0. with 10 mole % of CaF₂. It was mixed with TCP in a 2:3 ratio. FIG. 24 shows the XRD pattern before and after immersion.

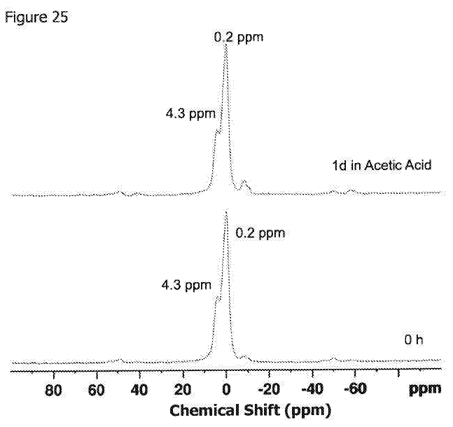

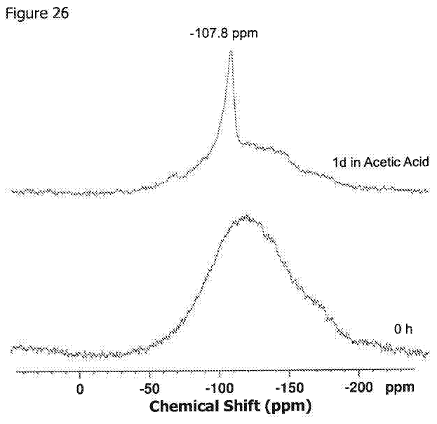

There is no apparent change in the XRD pattern before and after immersion with large amounts of TCP present after immersion. The ³¹P spectra are also shown (FIG. 25) no significant differences and virtually all the P is present as TCP. Some of the TCP has dissolved since the impurity peaks at −8.4 and 10.3 ppm are increased. FIG. 26 shows the ¹⁹F Spectra of the 2:3 mixture before and after immersion. Before immersion the ¹⁹F spectra exhibits a broad peak from −20 to −225 ppm with a broad maximum at about −100 ppm. Following immersion there is the formation of a peak at −107.8 ppm corresponding to CaF₂. It was expected that this glass wouldn't dissolve at all and all the fluorine would be present in an amorphous form. Less than about 20% of the fluorine in the glass has converted to CaF₂. However no fluorapatite is formed upon immersion with this glass. This glass gives the lowest pH rise in solution at 6 h and 24 h (FIG. 26) which is largely a result of its high NC.

### Example 8: A Fluorine Free Glass that does not Form Apatite

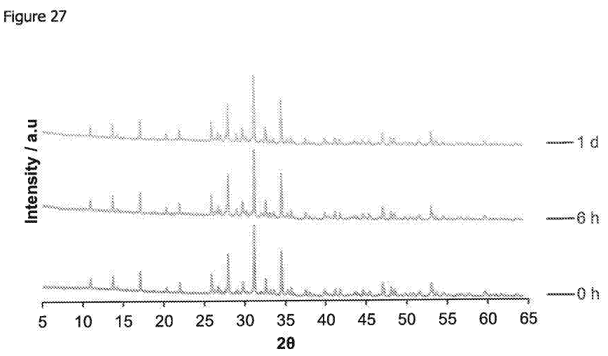

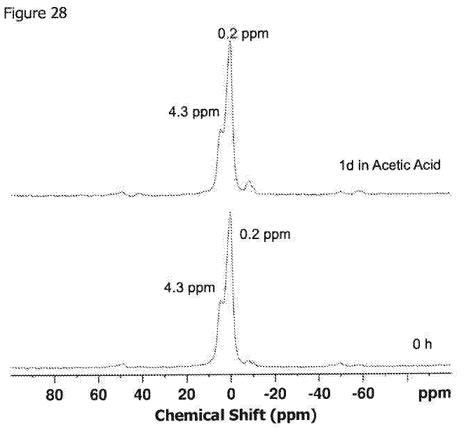

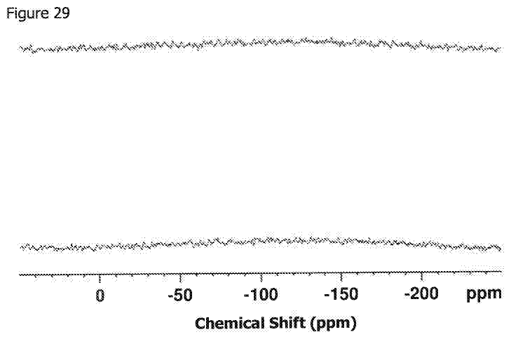

Glass 5 of Table 1 contains no fluorine. FIG. 27 shows the XRD of the 2:3 mixture with TCP. Only TCP is present. This is further confirmed by the ³¹P MAS-NMR spectrum after immersion for 24 h that shows only TCP (FIG. 28). The impurity peaks at about 8 and 10.3 ppm are enhanced slightly upon immersion indicating some dissolution of the TCP. The ¹⁹F spectra of the Glass 5/TCP mixture shows the glass to contain no F and no fluorine containing phases are present after immersion (FIG. 29). The pH of this mixture did increase which would be expected to favour the formation of apatite (hydroxyapatite as opposed to fluorapatite) but the pH rise was too low for this to occur in the absence of significant numbers of fluoride ions being released from the glass.

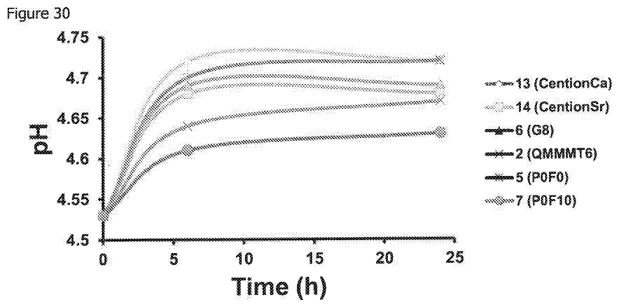

FIG. 30 shows the pH rises as a result of glass degradation for 2:3 ratio mixtures in 0.1 M acetic acid. Note all the glasses give a pH rise but glass 7 with a NC of 3.0 gives the lowest pH rise.

### Example 9 NaF/TCP Mixture

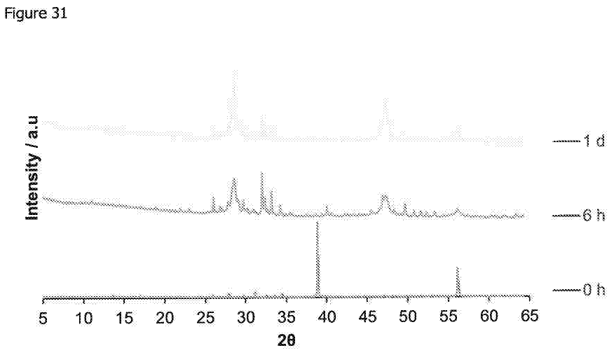

In this example rather than using a degradable glass as a source for fluoride release soluble sodium fluoride, NaF was used mixed with TCP in the 2:3 weight ratio. This equates to a fluoride concentration of 1260 ppm. This example was performed again in 0.1 M acetic acid at pH 4.5. FIG. 31 shows the XRD before and after immersion for 6 h and 24 h. The diffraction pattern of the initial mixture is dominated by the sharp diffraction lines of NaF at 38.9 and 56.1° two theta. The diffraction lines of TCP are present but are of low intensity relative to the NaF. Following immersion for 6 h both CaF₂ (diffraction lines at 28.4 and 47.1°) are present plus apatite diffraction lines in the range 30 34° two theta. After 24 h of immersion the apatite diffraction lines are much weaker and the diffraction lines for CaF₂ are stronger. This indicates that the FAP formed at 6 h is probably not stable and re-dissolves to form more CaF₂. Note that here because of the absence of glass the pH doesn't increase and the low pH favours CaF₂ formation at the expense of CaF₂.

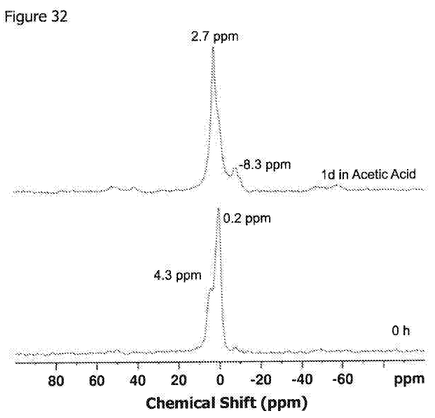

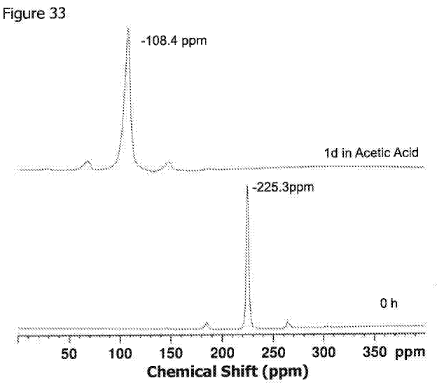

FIG. 32 shows the ³¹P MAS-NMR spectra before and after immersion. The sharp resonance at 2.7 ppm is indicative of apatite formation whilst the loss of the peaks at 4.6 and 0.2 ppm indicates dissolution of the TCP. The ¹⁹F spectra of the mixture before and after immersion is shown in FIG. 33. Before immersion the fluorine is present as crystalline NaF with a sharp peak at −225 ppm. This peak disappears upon immersion as the NaF dissolves.

The mixture forms largely CaF₂ with a chemical shift with no detectable Fluorapatite though it is probably present. It must be noted that there are two Fs per Ca in CaF₂ whilst only 0.2 Fs per Ca in Ca₁₀(PO₄)₆F₂ so the ¹⁹F spectrum is dominated by CaF₂.

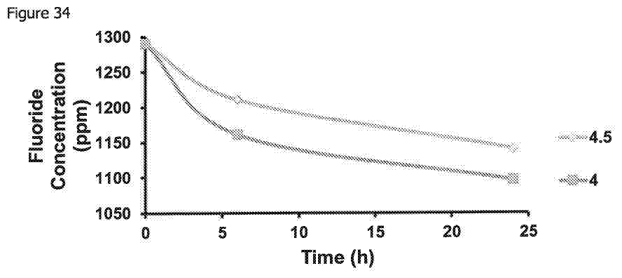

FIG. 34 shows the measured fluoride concentrations in solution of the mixtures with TCP at pH 4 and 4.5. The initial fluoride concentration reduces with time at pH 4 and 4.5. Fluoride is consumed in the formation of both CaF₂ and Fluorapatite. More Fluoride is consumed at pH4 than pH 4.5, which is probably a result of more CaF₂ forming.

The above described embodiments have been given by way of example only, and the skilled reader will naturally appreciate that many variations could be made thereto without departing from the scope of the invention.

### OTHER REFERENCES

Hill R and O'Donnell M “Multicomponent Glasses for Use in Personal Care Products” WO 2011/000866A2
R Hill, D. G. Gillam, A. J. Bushby, D. Brauer, N. Karpukhina and M. A. Mneimne “Bioactive Glass Composition” WO 2011/161422A1
Hill R G Collings A J Baynes I and Gillam D G “Multicomponent Oral Healthcare Composition” WO 2013/117913.
Mneimne M., Hill R. G., Bushby A. J. and Brauer D. S. “High phosphate content significantly increases apatite formation of fluoride-containing bioactive glasses”. Acta Biomater. 7 (2011) 1827-34.
Lynch E., Brauer D. S., Karpukhina N., Gillam D. G. and Hill R. G. “Multicomponent bioactive glasses of varying fluoride content for treating dentin hypersensitivity” Dental Materials 18 168-178 (2012)

1. A composition comprising a calcium orthophosphate and a bioactive glass comprising fluorine.

2. A composition according to claim 1, wherein the calcium orthophosphate is a tricalcium phosphate with a molar calcium to phosphorus ratio of 1.25:1 to 1.75:1.

3. A composition according to claim 1, wherein the bioactive glass has a fluoride content expressed as CaF₂ or SrF₂ of about 1 to about 30 mole percent.

4. A composition according to claim 1, wherein the bioactive glass has a fluoride content of about 5 to about 25 mole percent.

5. A composition according to claim 1, wherein the bioactive glass has a fluoride content of about 8 to about 23 mole percent.

6. A composition according to claim 1, wherein the composition comprises about 0.1% to about 15% by weight bioactive glass.

7. A composition according to claim 1, wherein the composition comprises about 0.1% to about 5% by weight bioactive glass.

8. A composition according to claim 1, wherein the composition comprises about 1% to about 3% by weight bioactive glass.

9. A composition according to claim 1, wherein the composition comprises about 0.1% to about 16% by weight calcium phosphate.

10. A composition according to claim 1, wherein the composition comprises about 0.1% to about 6% by weight calcium phosphate.

11. A composition according to claim 1, wherein the D50 particle size of the calcium phosphate is 12 microns or less.

12. A composition according to claim 1, wherein the D50 particle size of the bioactive glass is 12 microns or less.

13. A composition according to claim 1, wherein the D90 particle size of the calcium phosphate is 60 microns or less.

14. A composition according to claim 1, wherein the D90 particle size of the bioactive glass is 60 microns or less.

15. A composition according to claim 1, wherein the bioactive glass contains substantially no phosphate.

16. A composition according to claim 1, wherein the bioactive glass contains substantially no calcium oxide.

17. A composition according to claim 1, wherein the calcium orthophosphate is in the form of a calcium deficient apatite that contains substantially no hydroxyl ions.

18. A composition according to claim 1, wherein the calcium orthophosphate is in the form of a tricalcium phosphate

19. A composition according to claim 1, wherein the calcium orthophosphate is in the form of an amorphous calcium phosphate.

20. A composition according to claim 1 for use in the manufacture of a toothpaste.

21. A composition according to claim 20, wherein the toothpaste is a non aqueous toothpaste.

22. A toothpaste comprising a composition according to claim 1.

23. A composition according to claim 1 for use in the treatment or prevention of dental caries.

24. A method for making a toothpaste, which comprises the step of mixing a calcium orthophosphate and a bioactive glass comprising fluorine to formulate a composition according to claim 1.
